(19) United States
(12) Patent Application Publication
(10) Pub. No.: US 2005/0222539 A1
(43) Pub. Date: Oct. 6, 2005

(54) Automatic injection device

(75) Inventors: Gilbert R. Gonzales, New York, NY (US); Matthew J. Huddleston, Blacklick, OH (US); Kenneth E. Hughes, Pataskala, OH (US)

Correspondence Address: WOOD, HERRON & EVANS, LLP, 2700 CAREW TOWER, 441 VINE STREET, CINCINNATI, OH, 45202, US

(73) Assignee: PediaMed Pharmaceuticals, Inc.

(21) Appl. No.: 10/817,224

(22) Filed: Apr. 2, 2004

(51) Int. Cl. A61M 5/00

(52) U.S. Cl. 604/207

(57) ABSTRACT

An injector device comprising a body containing a syringe with a needle and plunger, a drive spring coupled with the syringe and operable, when released, to drive the syringe forward to inject the needle and subsequently to dispense a dosage from the syringe, a housing containing the body and drive spring, and a release apparatus coupled with the housing. The drive spring is initially locked in an unreleased position, and the body is slidable with respect to the housing and configured for sliding upward in the housing when the injector device is pressed down at an injection site to engage the release apparatus and release the drive spring for delivering a dosage.

### RELATED APPLICATIONS

This application is a continuation-in-part application of U.S. patent application ser. No. ______, entitled “Automatic Injection Device” and filed Mar. 30, 2004, under US Post Office Express Mail No. EV026538066US, which is incorporated by reference herein in its entirety.

### FIELD OF THE INVENTION

This invention relates generally to injection devices and specifically to automatic injection devices.

### BACKGROUND OF THE INVENTION

In certain medical situations, including potentially life-threatening situations, it has been a recognized procedure to provide a rapid and timely injection of an active pharmaceutical ingredient (API) in order to address a specific condition or symptoms. For such situation, single use, automatic injection devices are available. Automatic injection devices are self-powered, such as by an internal drive system, and will automatically inject a needle and dispense of dosage of an API upon being actuated or “fired.” Usually, a user need only hold the device at the injection site, fire it, and wait a short time for the injection. The device is then usually discarded. For example, APIs such as epinephrine and adrenaline are administered in such a fashion.

Oftentimes, the administration is on an emergency basis and the injection is directly into a suitable muscle, such as a thigh muscle. Existing automatic injection devices are usually self-contained and deploy rapidly and automatically (when fired) to deliver the dosage contained in the device. In one example, such a device may be used to administer a dosage of epinephrine for emergency treatment of serious allergic reactions or anaphylaxis, such as due to insect stings or bites, foods, drugs, or other allergens, as well as idiopathic or exercise-induced anaphylaxis.

Single-use injectors for such purposes are currently commercially available, with the EpiPen™ product being one such device. However, while existing products offer a convenient, self-contained and single-dose administration suitable for emergency uses, they have various drawbacks.

For example, the injection process itself is particularly vigorous, maybe even violent due to both actuation force and high puncture force. Furthermore, the existing injection devices are difficult to hold and orient at the injection site. The combination of the vigorous actuation and difficult handling sometimes makes a proper dosing difficult. In any case, it detracts from the comfort level of the patient, the injection administrator or both. Furthermore, because of the violent nature of the existing device, multiple injections are particularly undesirable. If only one hand of a patient or other person is available for using the device, this further exacerbates the problem. Thus, these devices require two-hand operation.

Furthermore, such existing devices can be difficult to actuate properly. While it is certainly desirable to avoid inadvertent actuation, once it is desired, the prior art devices can sometimes make it difficult to complete. In fact, it is often difficult to tell when the existing products are armed for injection. Still further, it is sometimes difficult to determine that they are properly oriented, and not reversed. An inadvertent reversal and firing of the device is extremely undesirable, and may be dangerous not only to a person administering the dosage, but also to the patient in need of it. Of course, this may be the same person in some emergency scenarios. Furthermore, the proper actuation of the device may be difficult to ascertain with existing products, adding further to the uncertainty of proper dosing.

With existing devices, it is also difficult to determine whether the injection process occurred correctly and whether a proper or a full amount of the dosage of the API has been dispensed to the patient.

Existing products utilize drive systems which act on the plunger of a syringe in the injection device to not only dispense the API dosage, but also to drive the syringe and needle for the purposes of an injection. The back pressure of the liquid API on the syringe plunger coupled with the friction between the plunger and syringe provides for the driving force to drive the needle. This presents difficulties if the needle encounters a greater resistance than normal. This may occur, for example, if multiple layers of clothes are passed through, the muscles at the injection sites are more tense than usual, or the needle strikes bone. In such a case, the needle may not penetrate properly into the skin and muscle at the injection site. Furthermore, the syringe plunger may begin to dispense the API dosage before the needle injection cycle is complete. Therefore, proper injections and proper dosages are sometimes suspect. Second injections, as noted, are undesirable, and may not even be possible unless an additional device is available.

Current products also instruct the user to wait a certain period of time (e.g., 10 seconds) once the auto-injection device is actuated, in order to ensure proper dosage amounts. However, in emergency situations, time references and elapsed time are often skewed. Furthermore, a parent or other person may be fighting with a reluctant child and will not have the ability to pay attention to a clock or watch during administration. Therefore, there is often a great amount of uncertainty regarding when the process is complete when using existing products.

Still further, the used injection device, which incorporates a needle, must be safely handled and disposed after usage. Existing products do not adequately address such issues. In some devices, the needle remains exposed after usage, thereby presenting a hazard. The EpiPen™, for example, requires the user to manipulate the needle after use, thus increasing the risk of needle stick wounds. Some injection devices have needle covers; however, they must be specifically deployed by the user after the injection device is used. Also, they are often retractable, so that some sticking/pricking hazard still exists.

As may be appreciated, such drawbacks of existing devices are even more highlighted in emergency situations where little time is available for reading literature, orienting the device, or just generally figuring out how the device works, checking to see that the injection is complete, and disposing of the used device. As a result, there is a need for a device that addresses the drawbacks of the prior art. There is further a need for an automatic injection device that is easy to operate and use and that provides a level of comfort to not only a patient, but also a person administering a dosage, in knowing that the injection was complete, the proper dosage has been administered and that the device may be readily and safely disposed of.

### BRIEF DESCRIPTION OF THE DRAWINGS

FIG. 1 is a perspective view of one embodiment of the invention in an armed state.

FIG. 1A is a cross-sectional view of the embodiment of FIG. 1.

FIG. 1B is a perspective view, partially cut away, of the embodiment illustrated in FIG. 1.

FIG. 1C is a side perspective view, partially cut away, of the embodiment illustrated in FIG. 1.

FIG. 2 is an exploded view of one embodiment of the invention showing body portions movable within a housing.

FIG. 2A is a cross-sectional view of an embodiment of the invention being fired to complete an injection stroke.

FIG. 2B is a perspective view, partially cut away, of the embodiment illustrated in FIG. 2A.

FIG. 2C is a side perspective view, partially cut away, of the embodiment illustrated in FIG. 2A.

FIG. 3 is an exploded view of the syringe subassembly of the present invention.

FIG. 3A is a cross-sectional view of an embodiment of the invention during the needle injection portion of the stroke.

FIG. 3B is a perspective view, partially cut away, of the embodiment illustrated in FIG. 3A.

FIG. 3C is a side perspective view, partially cut away, of the embodiment illustrated in FIG. 3A.

FIG. 4 is an exploded view of the drive subassembly of an embodiment of the present invention.

FIG. 4A is a side cross-sectional view of an embodiment of the invention during the dosage portion of the stroke.

FIG. 4B is a perspective view, partially cut away, of the embodiment illustrated in FIG. 4A.

FIG. 4C is a side perspective view, partially cut away, of the embodiment illustrated in FIG. 4A.

FIG. 5 is a perspective view illustrating components of the present invention.

FIG. 5A is a cross-sectional view of an embodiment of the invention with the protective sheath deployed after use.

FIG. 5B is a perspective view, partially cut away, of the embodiment illustrated in FIG. 5A.

FIG. 5C is a side perspective view, partially cut away, of the embodiment illustrated in FIG. 5A.

FIG. 6A is a perspective view of an alternative housing for the embodiment of the present invention.

FIG. 6B is a side perspective view of the embodiment illustrated in FIG. 6A.

FIG. 6C is a partial top view of the embodiment of the invention illustrated in FIG. 6A.

FIG. 7A is a side perspective view of a drive member of the present invention.

FIG. 7B is a top perspective view of a drive washer of the invention for use with the drive member of FIG. 7A, which is also shown in a top view.

FIG. 8A is a side perspective view of another drive member of the present invention.

FIG. 8B is a top perspective view of another drive washer of the invention for use with the drive member of FIG. 8A, which is also shown in a top view.

FIG. 9A is a side perspective view of another drive member of the present invention.

FIG. 9B is a top perspective view of another drive washer of the invention for use with the drive member of FIG. 9A, which is also shown in a top view.

FIG. 10A is a side perspective view of another drive member of the present invention.

FIG. 10B is a top perspective view of another drive washer of the invention for use with the drive member of FIG. 10A, which is also shown in a top view.

### DETAILED DESCRIPTION

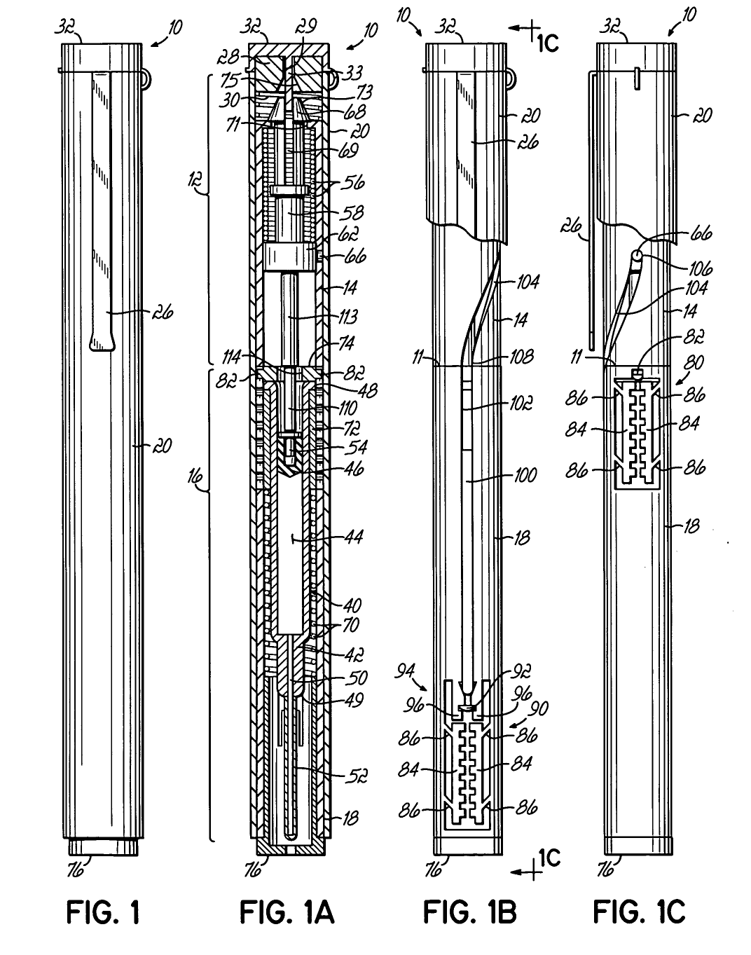

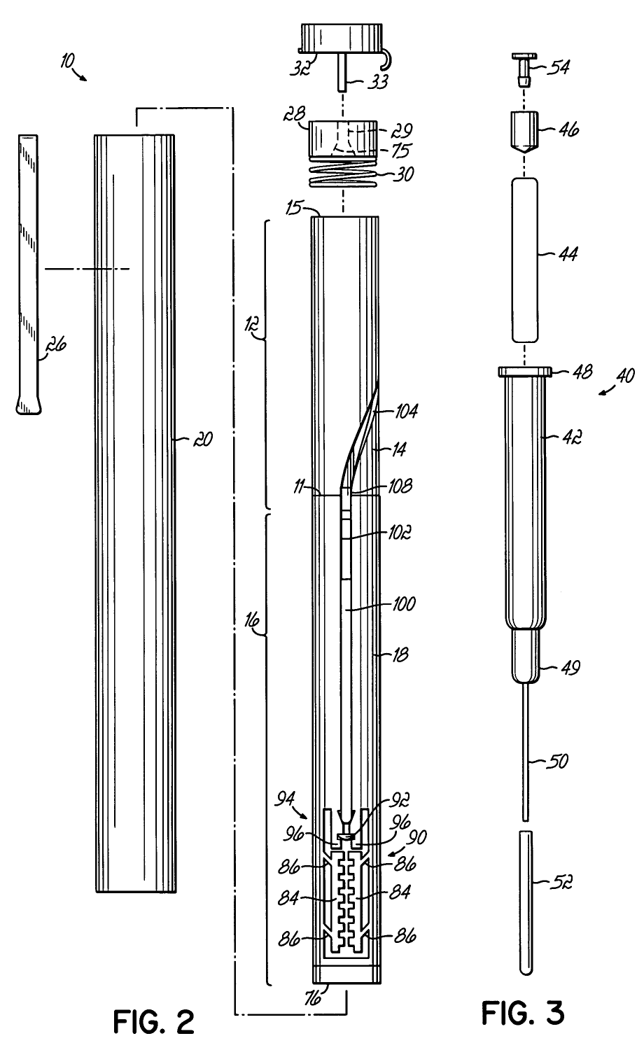

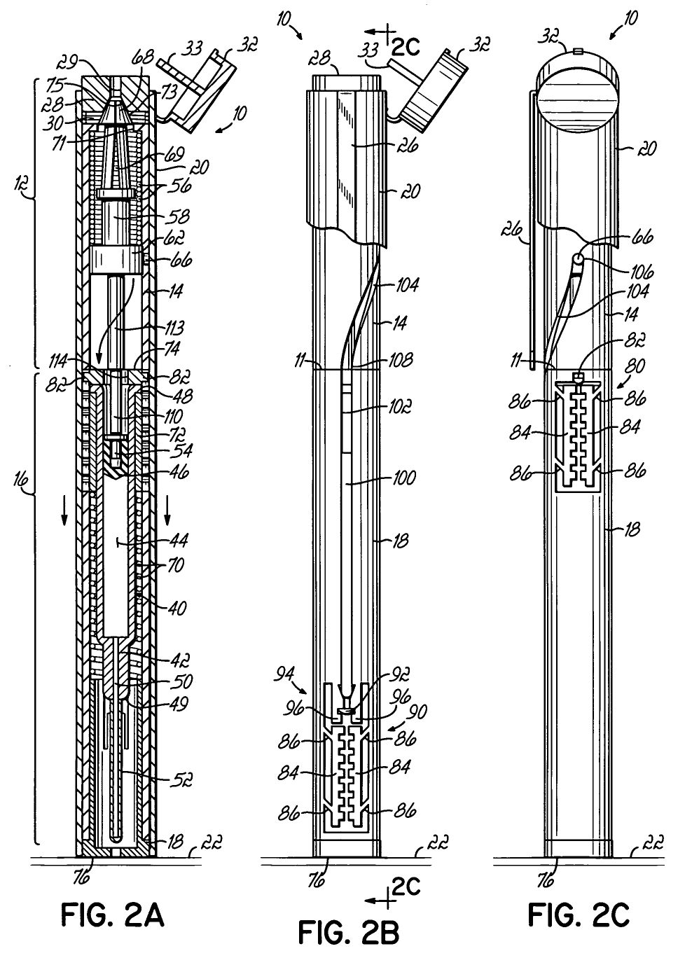

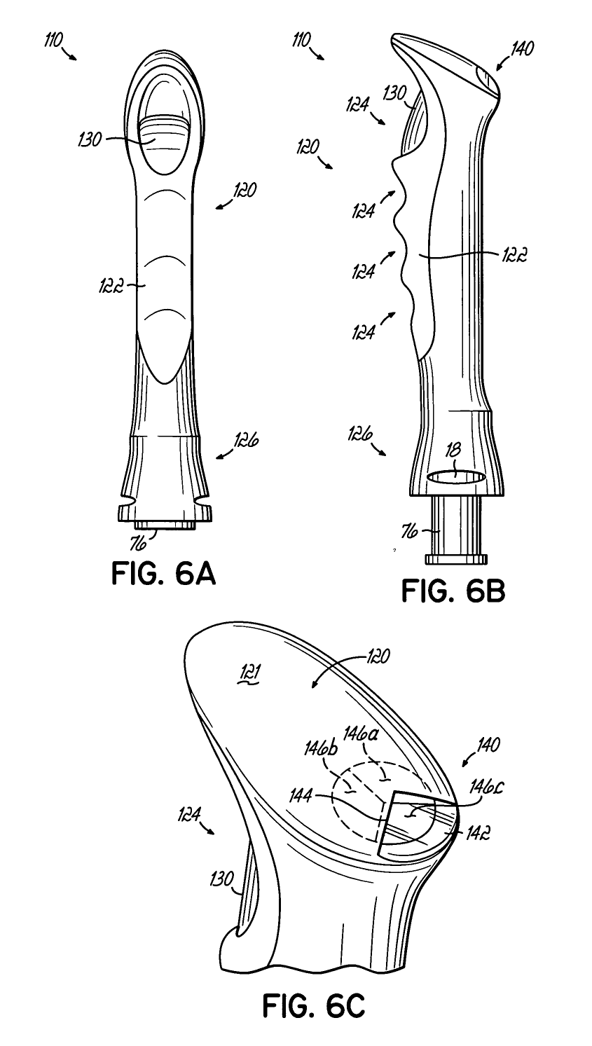

FIGS. 1-1C illustrate various views of one embodiment of an injector device incorporating various aspects of the present invention. In the embodiment illustrated in some of the Figures, the injector device has a general pen-like design, including an inner body or tube surrounded by an outer pen-shaped housing. Alternatively, other housings, as illustrated in FIGS. 6A-6C may be used. In the injector device 10, illustrated in the Figures, the inner body as shown in FIGS. 1B and 1C is divided into a plurality of body portions, primarily two sub-bodies, which house different subassemblies. The different sub-bodies contact or are coupled at line 11 and cooperate to act generally as a single body or body structure as described herein. Injector device 10 includes a drive subassembly 12, which includes the body portion or body 14, and an injector subassembly 16, which includes body or body portion 18. Although illustrated as individual bodies 14, 18, a unitary body structure for housing the different subassemblies may be utilized as well, in accordance with the principles of the present invention. The subassemblies 12, 16 are contained within an outer housing 20, which in one embodiment as illustrated in FIG. 1, may be tubular in form to overlie and telescopically engage bodies 14, 18. As noted, in accordance with one aspect of the present invention, the cooperating bodies 14, 18 act as a single body, which is generally slidable with respect to housing 20 and is configured for sliding upwardly in the housing when the injector device is pressed down at an injection site. The sliding movement of the body within housing 20 provides an engagement of a release apparatus to release a drive system for delivering a dosage of medicine from a syringe contained within the injector device 10. As illustrated in FIG. 1, the drive subassembly 12 is positioned on top of or above the injector subassembly 16. The injector device 10 is moved downwardly toward an injection site 22 (See FIG. 2A) to initially inject the needle and to subsequently dispense a dosage of the API or medicine from a syringe contained within the injector subassembly 16.

For explanatory clarity, the individual subassemblies are discussed individually herein below, followed by an overall discussion of the complete injector device illustrated in FIG. 1, and usage of that device.

Referring now to FIG. 2, the drive subassembly 12 and injector subassembly 16 are shown coupled together, such that the individual bodies 14, 18 for each of those subassemblies cooperate to form a single co-axial body to house the various elements of those subassemblies. An outer housing 20, in the form of a tubular housing in FIG. 2, is configured and dimensioned for containing the body of subassemblies 12, 16 therein. A clip 26 might be incorporated into the housing so that the injector device 10 resembles a pen. Also contained by housing 20, is a release apparatus 28, such as in the form of a release button 28. A release spring 30, in the form of a coiled spring, is also coupled with the release apparatus 28 and couples at one end with the drive subassembly 12. Alternatively, the release apparatus might be unitarily molded with the housing 20. Housing 20 is capped off at one end by a safety device, such as a safety cap 32, which operates to prevent inadvertent actuation of the injector device 10, as discussed further herein below. FIG. 1A illustrates the stacked engagement of the cap 32, release apparatus 28, and spring 30, with respect to the subassemblies 12, 16 inside housing 20. The housing 20, subassembly bodies 14, 18, release apparatus 28, and cap 32 may all be formed of a suitable material, such as a light-weight plastic. Spring 30 may be formed of a suitable resilient metal. The subassembly bodies 14, 18 of the device are configured to move coaxially and longitudinally in housing 20.

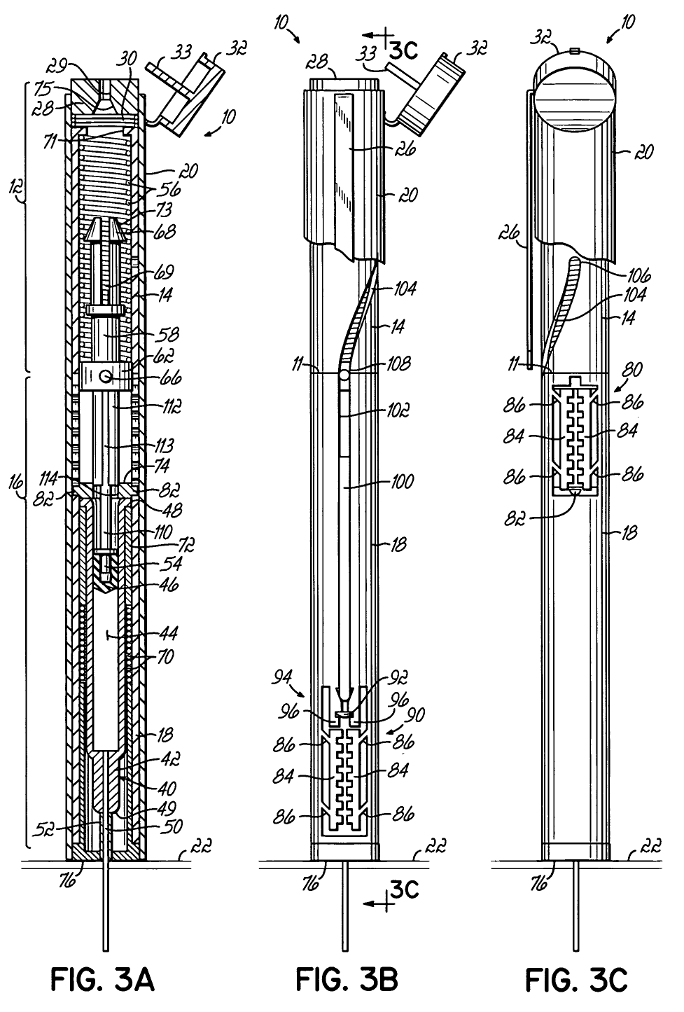

Turning now to FIG. 3, an exploded view of a syringe subassembly or prefilled syringe is illustrated. The syringe subassembly 40 is utilized in the injection subassembly 16, as illustrated in FIG. 1. Syringe 40 includes a syringe body or capsule 42, which contains a dosage of an active pharmaceutical ingredient (API) or medicine 44 to be injected into a user by the injector device 10. In one aspect of the present invention, the injector device 10 is used to administer a dosage of an API for emergency treatment of serious allergic reactions or anaphylaxis, such as due to insect strings, bites, foods, drugs, or other allergens, as well as idiopathic or exercise-induced anaphylaxis. For the administration of epinephrine, for example, the syringe body 42 might be configured to hold a dosage amount in the range of 0.01 mg/kg every 15 minutes in children and 0.2-0.5 mg/kg every 20 minutes in adults (0.15 mg of 1:2000 [2 mL] in children by intra-muscular auto-injector and 0.3 mg of 1:1000 [2 mL] in adults by intra-muscular auto-injector). Alternatively, other APIs might be utilized to address various other medical situations. For example, the syringe 40 may contain antihistamines, bronchodilators, analgesics, narcotics, analeptic agents, anesthetics, anticonvulsants, antihypertensives, anti-infectives, antidiabetic agents, antidotes (e.g., anticholinergic antagonists, anticholinesterase antagonists, antivenins, benzodiazepine antagonists, chelating agents, digoxin antagonists, narcotic antagonists, nondepolarizing muscle relaxant antagonists) antiemetics, anti-inflammatory agents, antiparkinsonian agents, asthma agents, antipsychotic agents, bronchodilators, cardioprotective agents, cardiovascular agents, central nervous system stimulants, detoxifying agents, vascular dilators, antihypoglycemic agents, antihyperglycemic agents, mixed opioid agonist/antagonists, insulin, hormones, migraine management drugs, motion sickness products, parasympatholytics, parasympathomimetics, psychotherapeutics, respiratory agents, sedatives and hypnotics, diphenyhydramine, albuterol, bitolteride, terbutaline, phenergan, hydroxyzine, prednisone, prednisolone, dexamethazone, methylprednisolone, nitroglycerin, cortisone, morphine, codeine, fentanyl, salbutamol, ipratropium, bromide, theophylline, aminophylline, fluticasone, budesonide, beclomethasone, and glucagon.

The syringe barrel or body 42 may be any suitable syringe body available for the purposes of injection. It may be either a standard size, or it might be custom designed for the purposes of the injector device 10. Syringe 40 also includes a plunger 46 to be driven through the syringe body 42 to dispense the API 44. As is typical, the plunger is initially positioned at the proximal end 48 of the syringe body and then is driven along the length of the syringe to dispense the API 44 through a needle 50. In one embodiment of the invention, a needle of any suitable size may be used. For example, needle size range of 14 gauge to 30 gauge (with usual needle range of 21 gauge to 25 gauge, in lengths of 15 mm to 25 mm, may be suitable, although other sizes might also be utilized. The needle is rigidly mounted to the distal end 49 of the syringe body as is typical. For the purposes of sterility, needle 50 may be initially covered by a needle boot 52, which is generally formed of a thin, flexible plastic or rubber material, which may be easily penetrated by the needle during the injection portion of the operation of injector device 10. The needle boot 52 may perform the secondary function of sealing the needle tip to prevent leakage of the API 44 from the prefilled syringe 40.

For dispensing the API 44, plunger 46 is driven by the drive system of the drive subassembly 12. To that end, the plunger 46 is coupled with a plunger adaptor 54, which interfaces with a component of the drive subassembly 12. Plunger 46 is formed of a resilient rubber or plastic and fits snugly within the syringe body 42 to prevent leakage and to dispense the API when the plunger is driven in the syringe body. The syringe body 42 and or plunger 46 may be coated with silicone, Teflon™, or other friction-reducing agents.

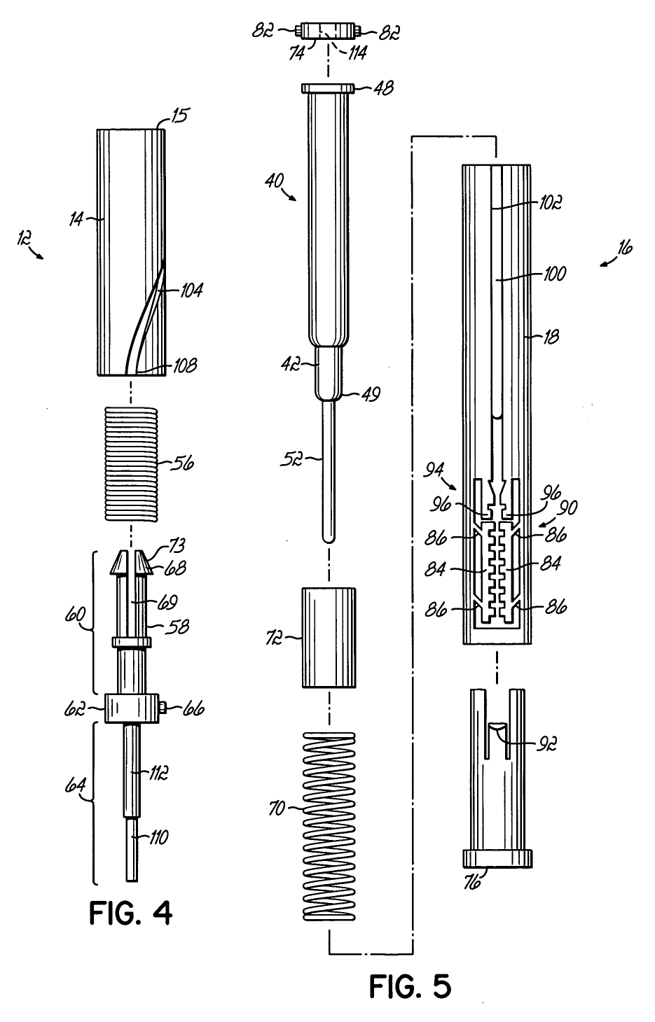

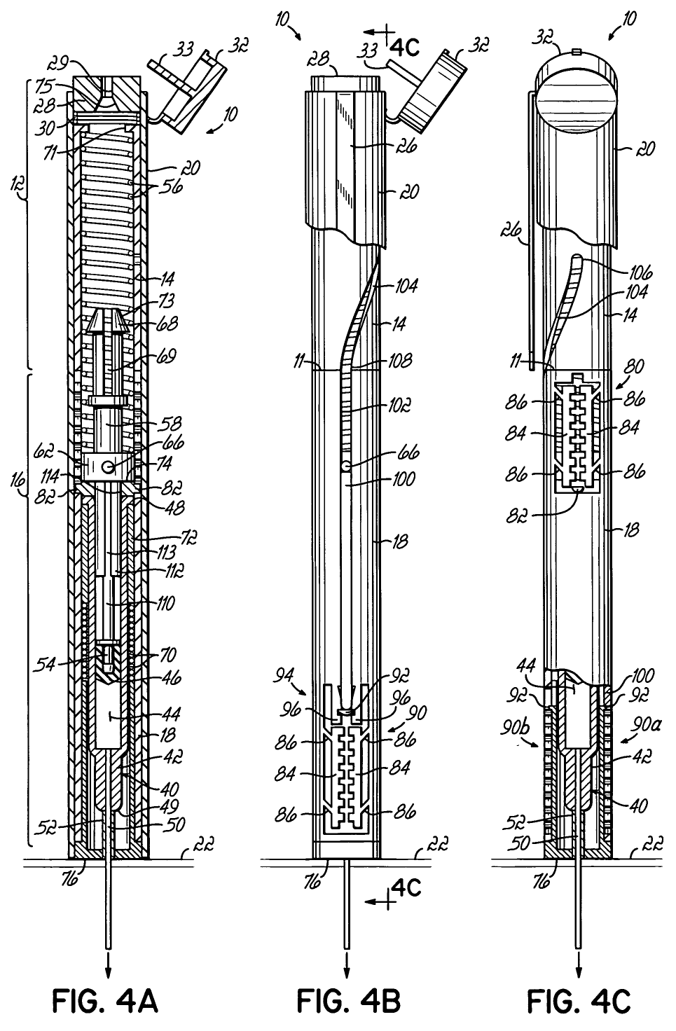

Turning now to FIG. 4, the drive subassembly 12 is illustrated and includes the body or body portion 14, shown in the illustrated embodiment as a tube, and a drive system including a drive spring 56 and a drive member 58 driven by the drive spring. The drive subassembly 12 provides a drive system, which essentially has a stroke that acts in multiple ways on the syringe subassembly 40 to first inject the needle 50 and then drive the plunger 46 forward to dispense the API through the injected needle. The stroke of the drive system 12 is defined by the extension of spring 56 and the movement of drive member 58. In accordance with one aspect of the present invention, the stroke essentially has an injection portion or segment, wherein the syringe is driven forward to inject the needle, and a dosage portion or segment, wherein the plunger is driven forward to dispense a dosage from the syringe.

Referring to FIG. 4, drive member 58 is a bifurcated member and includes a bifurcated section 60, which is split along its longitudinal axis by slot 69 to form two different sides or halves, as illustrated in FIG. 4. A collar 62 couples the bifurcated section with a rod section 64, which drives the syringe and plunger, as discussed further herein below. Guide collar 62 includes a key structure 66 for guiding the drive member as it is driven by spring 56 within slot 104 in body 14 and slot 102 in body 16.

As illustrated in FIG. 4, the bifurcated section 60 fits inside or extends through spring 56 and the spring 56 is therefore captured between a proximal end 15 of the body 14 and drive collar 62. In that way, when the spring is released, it drives the drive member 58 downwardly to dispense a dosage from the syringe. Initially, within the drive subassembly 12, the drive spring is locked in a compressed and unreleased position. That is, the drive member 58 is locked with respect to body 14 to compress the spring 56 therebetween (See FIG. 1A). To facilitate the initial locking of the spring in an unreleased position, the bifurcated drive member 58 includes opposing shoulders 68 at one end of the bifurcated section 60. The shoulders 68 fit appropriately through an opening 71 in the end 15 of the body 14. The resiliency of the bifurcated section 60 maintains the shoulders apart, separated by the slit 69 of the bifurcated section. The shoulders extend through and engage an end of the body 14 (See FIG. 1A), and thus maintain the drive member 58 up in the body with the spring 56 compressed, and locked in an unreleased position. The shoulders 68 are tapered to form what might be considered a cam surface 73. The cam surface 73 interacts with the corresponding cam surface 75 in the release apparatus 28 for releasing the drive member and spring 56.

Referring again to FIG. 2, the release apparatus 28 includes a tapered indent 29 formed therein, which is configured to cooperate with the tapered shape of the shoulders 68. The tapered indent 29 essentially forms a corresponding cam surface 75, which acts on the cam surface of the shoulders to drive the shoulders in the two sides of the bifurcated section 60 together, when the end of the drive member 58 is pushed upwardly against the release apparatus 28.

In accordance with another aspect of the present invention, the overall body of the mechanism, which is made up of body 14 of the drive subassembly, and the body 18 of the syringe subassembly is slidable with respect to the housing 20, and is configured for sliding upward in the housing when the injector device is pressed down at an injection site. The sliding body, in effect, engages the release apparatus 28, coupled with the housing 20, to release the drive spring for delivering a dosage. More specifically, the sliding of the body 14, 18 moves the shoulders 68 up into the aperture 29 of release apparatus 28, and the cooperating cam surfaces 73, 75 drive the bifurcated sections 60 together, such that the shoulders 68 are now unlocked and they slide through the opening 71 in drive body 14, and thereby release the force of drive spring 56 on the drive member 58.

Referring again to FIG. 1, a portion of a protective sheath 76 extends out of the distal end of housing 20 when the device 10 is armed for use. When injector device 10 is positioned at an injector site 22 and pushed down on a surface (See FIG. 2A), such as the skin surface, the sheath 76 acts against the body 18, and thereby the body 14, so they both slide upwardly to drive the shoulders 68 of the bifurcated drive member 58 to engage the release apparatus 28. The release apparatus squeezes the sides of section 60 together to release the spring 56. Therefore, the injector device 10 will not “fire” or is not actuated until it is pushed down on the injection site. The injector device of the present invention thus includes a safety feature that will prevent the inadvertent firing of the device until the user actually positions it at a suitable injection site and then pushes downwardly on the device with a certain force at the site.

In accordance with another aspect of the present invention, another safety feature is provided by a safety device, which engages the bifurcated drive member 58 and prevents it from being squeezed together. For example, safety device 32, which may be hingedly coupled, removably coupled or otherwise movable, with respect to housing 20 includes a post or other suitable structure 33, which extends through the release apparatus 28, through aperture 29, and thereby engages the bifurcated member 58. Referring to FIG. 1, the post 33 fits into the slot 69 of the bifurcated section 60 and thereby maintains the shoulders 68 separate from each other. To prevent member 58 from passing through opening 71. Device 32 also generally prevents inadvertent firing of the injector device until the post is removed from slot 69. Therefore, in sequential order, the post 33 is removed (e.g. cap flipped up) to arm the device and then the end of the injector device 10 would be pushed downwardly onto an injection site in order to drive the body up into housing 20 to engage the bifurcated member 58 with the release apparatus 28 and thereby release spring 56. This facilitates the injection.

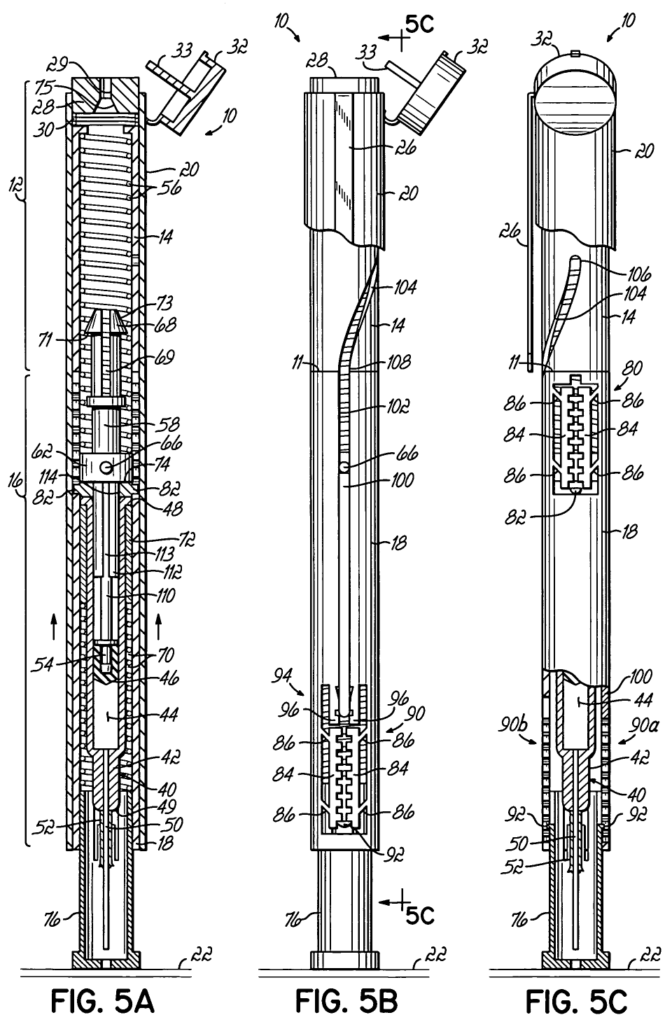

Turning now to FIG. 5, the injector subassembly 16 incorporates the syringe subassembly 40 therein, and includes the body portion or body 18 to house the syringe subassembly and other components. The syringe subassembly 40 is contained co-axially within the body 18, along with a sheath spring 70 and a syringe spacer 72. As illustrated in FIG. 1A, the syringe 40 extends inside the spacer 72 and spring 70. The syringe spacer 72 is captured between the distal end 48 of the syringe body 42 and the sheath spring 70 to compress the sheath spring, as discussed further herein below. Positioned on top of the distal end 48 of the syringe 40 is a drive washer 74, which interfaces with the drive member 58 and acts against the syringe 40. A protective sheath 76 is positioned inside the distal end of body 18 to telescopically move therein, as shown in FIGS. 5A-5C, and discussed further herein below.

Referring to FIG. 1C, body 18 of the injection subassembly includes a ratchet structure 80 formed therein. In one embodiment, there are two ratchet structures on body 18, approximately on opposite sides or 180 degrees apart. The individual bodies, or body portions 14, 18, may be formed of a suitable material such as plastic and, thus, the ratchet structure 80 might be molded into the plastic as shown. The drive washer 74 includes opposing posts 82, which each engage a corresponding ratchet structure 80. The ratchet structures might be duplicated on opposing sides of body 18 or a single ratchet structure (single post 82) might be utilized. As illustrated in FIG. 1C, the illustrated embodiment of the ratchet structure includes opposing rows 84 of ratchet teeth. The rows 84 cantilever out from the body on resilient legs 86 to allow the rows to be spread apart, as the washer is driven and the post 82 moves along the ratchet structure. Specifically, as shown in FIG. 3C, when the drive washer 74 and syringe are driven downwardly in the injection portion of the stroke, the posts 82 on washer 74 ratchet down between the rows of teeth 84 of ratchet structure 80 so that, as the needle is injected, the needle remains at the furthest ejected position with respect to the injector device housing 20 to thereby prevent the needle from being pushed back up into the housing. This insures a more certain injection stroke and proper injection depth or needle placement of the injection needle in the tissue before the dosage is dispensed. As shown in the side view of FIG. 1C, the post 82 is generally semi-circular with a curved side facing downwardly and a flat side facing upwardly. In that way, the curved side can act to spread the cantilevered rows 84 of the ratchet structure apart as the drive washer 74 moves downwardly. Thereby, the posts 82 ratchet along the rows of teeth 84. Subsequently, the flat upper side of the post engages the previous teeth of each ratchet increment to prevent the drive washer from being retracted or pushed upward in the body 18. As shown in FIG. 3C, at the completion of the injection portion of the stroke, the post is at the bottom of the ratchet structure 80 and is held there by the ratchet structure. The downward angle of the legs 86 ensures that the rows 84 cantilever such that the post may push them away from each other to allow the post 82 to ratchet downwardly in the downward injection stroke, but then compress toward each other as to lock, preventing travel of the post and drive washer in the opposite or upward direction.

Referring now to FIG. 1B, a similar ratchet structure 90 might also be formed in body 18 for the protective sheath 76. A single ratchet structure might be used, or preferably, an additional ratchet structure is also formed on the other side or 180 degrees apart. The opposing ratchet structures are indicated as 90a and 90b in FIGS. 4C and 5C. The protective sheath also includes posts 92 on either side, which engage the corresponding ratchet structures. As illustrated in FIG. 1B, the post 92 on at least one side is initially engaged by a latch structure to prevent the protective sheath from being released. The embodiment of the latch structure 94, illustrated in FIG. 1B, includes two opposing spring fingers 96, which work together to capture post structure 92, and thereby prevent the sheath from being released and deployed until an appropriate time in the injection sequence, discussed further herein below. To actuate deployment of the protective sheath 76, injector device 10 includes a slide 100 that is coupled to move in an elongated slot 102, formed in body 18. The slide 100 may be slid downwardly to engage the latch structure 94 and thereby release the protective sheath, as illustrated in FIGS. 4B and 5B and discussed further herein below.

Body 14 also includes a slot 104 formed therein to wrap at least partially around the body from a first position 106, as illustrated in FIG. 1C. The slot extends between a first position 106 and a second position 108 as illustrated in FIG. 1B. The slot 104 of the illustrated embodiment is configured to generally rotate approximately 90 degrees around the body 14. The second position 108 coincides with the top of slot 102 and, thus, when the body portions 14, 18 are positioned together within housing 20 to form a singular body structure, the slots 104 and 102 align. The key structure 66 on collar 62 of the drive member 58 follows the slot 104, and then slot 102 during the injection portion and dosage portion, respectively, during the stroke of the injector device 10. In one aspect of the present invention, the drive member 58 rotates from the first position 106 to the second position 108 in the stroke and drives the washer 74 to drive the syringe and needle forward. Upon reaching the second position, the drive member passes through the washer and then drives the plunger forward to dispense the dosage.

Referring again to FIG. 4, the injection section 64 of the drive member 58 includes a forward section 110, which extends through drive washer 74 to engage plunger 46 and, specifically, to engage the plunger adaptor 54 (See FIG. 1A). The second section 112 acts upon the drive washer 74 during the injection portion of the stroke. Section 112 includes a key slot 113 formed therein (see FIG. 3A), which, when the member 58 is fully rotated, engages an appropriate key 114 on the washer (See FIG. 5), which then travels in slot 113. This allows section 112 of the drive member 58 to pass though the washer 74 and thereby drive the plunger 46 downwardly, so the syringe body 42 dispenses the API 44 through needle 50.

To illustrate operation of the injector device 10, a sequence of events illustrating the stroke of the device, as well as the position of various of the components, is discussed progressing from FIGS. 1A-1C through to FIGS. 5A-5C. FIGS. 1-1C illustrate the device in the loaded and locked state, wherein the injector device is ready for use and actuation and the drive system is locked in an unreleased position. This is generally the state in which the injector device will be carried, such as in a pocket or purse. Referring to FIG. 1A, the drive spring 56 is shown compressed between the collar 62 of the bifurcated drive member 58 and the top of body 14. Shoulders 68 of the bifurcated drive member 58 extend through an appropriate opening 71 formed in the top of body 14. The body 14, 18 that is slidable within the outer housing 20 is biased downward by spring 30 and the release apparatus 28, which is coupled with or may be part of housing 20. As shown in FIG. 1A, the post 33 extends into the slot 69 formed between the two portions of the bifurcated member 58 and the bifurcated shoulders 68 to prevent those shoulders from coming together and passing through opening 71. As such, the body 18 is prevented from sliding into the housing to fire or actuate the injector device.

As illustrated in FIG. 1B, the slide 100 is in its uppermost position, and the key structure 66 of the drive member 58 is in the first position 106. The protective sheath 76 is prevented from being released out of the end of body 18 by the latch structure 94. The protective sheath does not engage ratchet structure 90. Nor does post 82 of the drive washer 74 engage the ratchet structure 80. In FIGS. 1B and 1C, the housing 20 is shown cut away to show the ratchet structures 80, 90 and the slots 102, 104. When it is desirable to deploy the injector device of the invention, the post 33 is removed from between the bifurcated member.

Turning to FIGS. 2A-2C, FIG. 2A illustrates device 32, hinged back to remove post 33 from the slot 69 of the bifurcated member. Of course, if cap 32 is a separate piece, it might simply be removed. Upon positioning the injector device 10 at an injection site 22, the injector device 10 is pushed downwardly by a user to push the sheath 76 and the end of body 18 up into the housing 20. The exposed end of sheath 76 shown in FIG. 1A is pushed up into the housing (FIG. 2A) against the bias of spring 30. Spring 30 may be configured to present a biasing force of approximately 1 to 5 pounds. In that way, a similar downward force on the housing (upward force on body 18, 14) would be required by the user at the injection site to overcome the spring force of spring 30. In doing so, the drive member 58 and specifically the shoulders 68 are driven up into aperture 29 of the release apparatus 28. Due to the cooperating cam surfaces 73, 75, the shoulders 68 and the bifurcated section 60 of drive member 58 are pushed or squeezed together so that the bifurcated member can pass through the opening 71 formed in the top or the distal end 15 of the body 14. In that way, the drive member 58, under the forceful bias of the drive spring 56 against collar 62, will be driven downwardly as part of the stroke and against the drive washer 74. As the drive member 58 travels downwardly, it also rotates as the key structure 66 follows slot 104. More specifically, when the body slides upward into the housing as the injector device is pressed down at an injection site, the drive member engages the release apparatus and releases the drive spring for beginning the stroke of the drive member 58 for delivering the dosage.

Referring now to FIGS. 3A-3C, the injection portion of the stroke is illustrated. The released drive spring 56 acts on collar 62 and thereby drives the drive member 58 and washer 74. In turn, the drive member 58 is coupled with the syringe body 42 through drive washer 74. Drive washer 74 has a greater diameter than the diameter of the upper or proximal end of the syringe body 42. As such, downward movement of drive member 58 translates into a downward movement of the syringe, thus driving the needle 50 through the needle boot 52 and out of the end of the protective sheath 76 and into the injection site, such as the surface of a user's skin. The sharp needle is driven in through the skin and into the muscle at the injection site. Preferably, the needle first pushes through the flexible needle boot 52 that then bunches up inside the sheath 76. The needle then penetrates the skin.

The drive spring is configured to provide a sufficient force to drive the syringe and needle forcefully into the injection site. Preferably, the spring provides such a sufficient driving force without an overly forceful amount of shock to the user. In one embodiment, a needle providing an injection force in the range of 0.25 to 10 pounds of force would be suitable. As noted below, the force of spring 56 must overcome the force of spring 70 to deploy the sheath 76.

Section 112 of the drive member 58 engages drive washer 74 but does not pass therethrough until the drive member 58 and collar 62 are rotated completely to the second position 108, as illustrated in FIG. 3B. At the second position, the washer key 114 slides in the key slot 113. As such, the effective length of the injection portion of the stroke is determined by the vertical length of the slot 104 between the first position 106 and the second position 108. As the drive member 58 drives the drive washer 74 and syringe 40 downwardly, the drive member rotates, guided by slot 104. Simultaneously, the posts 82 on the drive washer engage the ratchet structure 80 during the injection portion of the stroke, pushing through the cantilevered rows of teeth 84 at various ratchet intervals to lock the syringe in various sequential injected positions. Once the post 82 has begun ratcheting within the ratchet structure 80, it can generally travel only in the downward direction.

Referring to FIG. 3C, upon the drive member and key structure 66 reaching the second position 108 as indicated by key structure 66 in FIG. 3B, the post 82 is in the downwardmost position with respect to the ratchet structure 80. This locks the syringe in the fully injected position, as shown in FIG. 3C. The key structure 66 generally follows the entire rotational length of slot 104 until it reaches the second rotational position 108. When the drive member 58 has progressed to the second position, section 112 of the drive member, having a key slot 113 formed therein, aligns with the key structure 114 in the drive washer 74, as illustrated in FIG. 3A. With such a keyed alignment, the drive member is then able to pass through the drive washer 74, which is at its downward most position in order to act on the plunger 46 and thereby drive the plunger 46 and adaptor 54 downwardly to dispense the API. More specifically, the section 112 of the drive member 58 passes through washer 74. In that way, further downward movement of the member 58 (the dosage portion of the stroke) is used to dispense the dosage of the API. The portion of the stroke indicate in FIG. 3A-3C is generally indicated as the injection portion of the stroke or the injection stroke.

Therefore, in accordance with one aspect of the invention, the dosage is not dispensed until injection into the body of the needle reaches the full extension in the muscle (e.g., 15 mm). This eliminates a resistance-actuated injection that may occur with prior devices when the needle encounters bone or clothing. The present invention directs its force against the syringe rather than against a liquid dosage.

Generally, the drive member 58, in an injection portion of the stroke, drives the drive washer 74 to drive the syringe, and, in a dosage portion of the stroke, passes through the drive washer 74 to drive the plunger 46. The disclosed embodiment of FIGS. 1-5 illustrates an embodiment that utilizes generally a 90-degree rotation of the drive member 58 along slot 104 to provide a keyed alignment, which ensures a complete injection portion of the stroke followed by the dosage portion of the stroke. However, other alternative embodiments might provide greater or lesser rotations for the drive member to pass through the drive washer. Furthermore, other various shapes or interactions might be provided for the drive member 58, and particularly the injection section 64 (i.e. section 112) of the drive member 58, as well as the key structure or opening 114 in the drive washer 74, in order to achieve this aspect of the present invention.

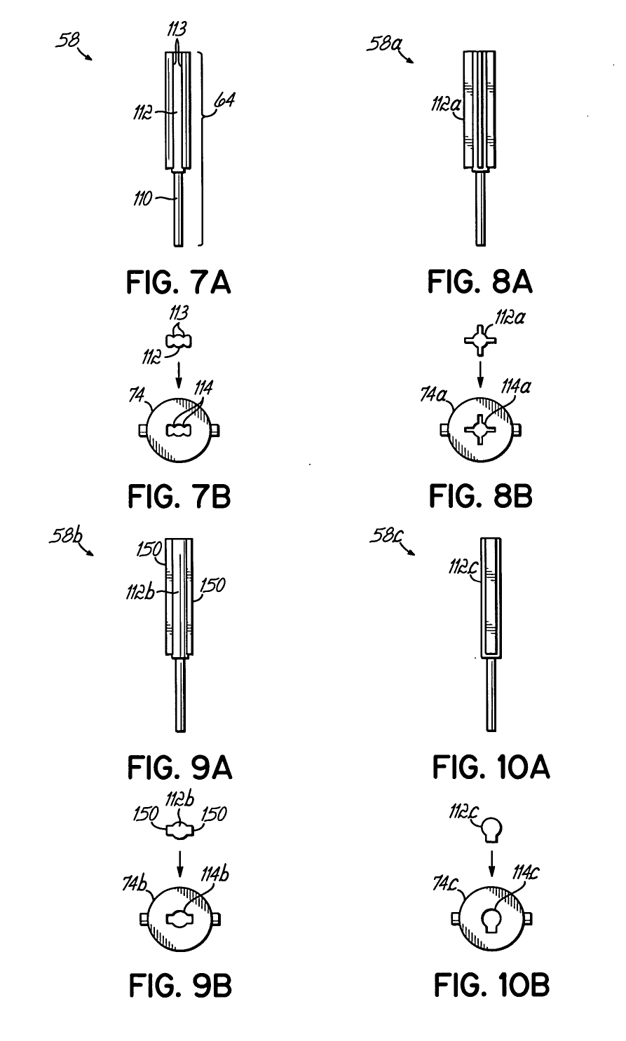

For example, referring to FIG. 7A, the injection section 64 of the drive member 58 is illustrated with section 112 and key slot 113. FIG. 7B illustrates the corresponding key structure 114 for the drive member 58. While a 90 degree rotation is used, because of the shape of the key structure 114 and key slot 113, a rotation of close to 180 degrees might by utilized as well before the key slot 113 and key structure 114 would align for the section 112 to pass through the washer 74.

FIGS. 8A-8B, 9A-9B and 10A-10B illustrate alternative drive members and drive washers. For example, the drive member 58a of FIGS. 8A-8B has a section 112a that has a generally cross-like shape in cross section. The corresponding key structure, opening, or aperture 114a of the drive washer 74a may then have a cross-sectional shape to match. The embodiment of FIGS. 8A and 8B will allow around a 90 degree rotation only before passing through the drive washer.

In FIGS. 9A and 9B, the drive member 58b has a section 112b which has an elongated shape in cross section formed by a circular portion having opposing side ribs 150 extending outwardly therefrom. The corresponding key structure, opening, or aperture 114b of the drive washer 74b could then have a somewhat similar shape to match. The embodiment of FIGS. 9A and 9B will allow around a 180 degree rotation similar to that embodiment illustrated in FIGS. 7A-7B before passing through the drive washer.

The drive member 58c of FIGS. 10A-10B might have a section 112c that has a generally circular shape in cross section with a rib 152 extending outwardly therefrom. The corresponding key structure, opening, or aperture 114c of the drive washer 74c could then have a cross-sectional shape to match. The embodiment of FIGS. 10A and 10B will allow around a 360 degree rotation before passing through the drive structure. As will be appreciated, the possible angles of rotation will not be exactly 90, 180 or 360 degrees, for example, because it will be desirable to offset the drive member and drive washers from each other a significant extent to prevent the drive member from inadvertently passing through the washer until it is rotated the desired amount. Furthermore, any angular rotation might be utilized based on the shapes of the drive member and washer and their initial orientation with respect to each other.

Furthermore, it is not necessary that the cross section of a portion of the drive member match the shape of the opening in the drive washer, although the illustrated embodiments have this correlation. Rather it is generally desirable, in accordance with the principles of the invention, to have the drive member drive the washer during one portion of the stroke and then pass through the drive washer in another portion of the stroke. This might be accomplished with other shapes as well for the drive member and drive washer as taught herein without deviating from the invention.

FIGS. 4A-4C illustrate the remaining portion of the stroke or the dosage portion of the stroke. Referring now to FIG. 4A, the drive member 58 and section 112 thereof is illustrated as passing through the drive washer 74. Section 112 passes through the drive washer 74 during the dosage portion of the stroke and thereby drives the plunger 46 into the syringe body 42 to dispense the API. The length of section 112 of the drive member essentially defines the movement of plunger 46 and dosage portion of the stroke. The overall length of the drive member may be varied for varying the dosage amount driven out of the syringe by the plunger 46. The drive spring 56 continues to drive member 58 downwardly until collar 62 engages the drive washer 74. Referring to FIG. 4B at the second position, drive member 58 no longer rotates as the key structure 66 of the collar follows along slot 102. As the drive member progresses through the dosage portion of the stroke and generally through the completion of the dosing portion, the drive member 58, via key structure 66, then engages the slide 100. Referring to FIG. 4B, the slide 100 moves or slides longitudinally downwardly in slot 102 at the urging of drive member 58 and key structure 66. The key 66 acts on the proximal end of the slide and pushes it until the distal end of the slide engages the latch structure 94.

Turning to FIG. 3B, during the injection portion of the stroke and previous to that, the opposing arms 96 cooperate to capture the posts 92 of the protective sheath. This keeps the sheath locked. However, as illustrated in FIGS. 4B and 5, the slot 102 tapers at a position 103 proximate the latch 94. When guide 100 engages the tapered position 103, it is configured to drive the arms 96 apart and thereby release the posts 92, thus releasing the protective sheath for deployment. That is, generally upon completion of the dosage portion of the stroke when the full or proper dosage amount is dispensed, the protective sheath 76 is released and ready to cover the needle when the injection process is complete. The full dosage amount refers to the desired full stroke of the plunger and not necessarily that all of the API in the syringe is dispensed. There may still be some residual API.

Referring to FIGS. 2A and 3A, during the injection portion of the stroke, the sheath spring 70 is compressed and loaded to thereby bias the protective sheath downwardly. Turning to FIG. 2A, the sheath spring 70 is illustrated in a generally uncompressed state and is captured between a top or proximal end of the protective sheath 76 and the syringe spacer 72. The spacer 72 is positioned between the top of the sheath and the top end of the syringe body 42.

Referring then to FIG. 3A, as the injection portion of the stroke continues, the syringe body 42 and syringe spacer 72 are driven downwardly to compress the sheath spring 70 against the protective sheath 76. Therefore, upon completion of the injection portion of the stroke, the sheath spring is compressed and loaded, and the sheath acts upon the protective sheath to urge it out of the body to cover the needle. The protective sheath is latched or locked into place by interaction between the latch 94 and the posts 92 of the protective sheath.

Turning now to FIG. 4B, when slide 100 engages the latch 94, the posts 92 are released, thereby releasing the protective sheath 76 for deployment.

Turning now to FIGS. 5A-5C, when the injector device 10 is moved away from the injection site 22, the protective sheath 76 automatically moves forward to cover needle 50 under the force of sheath 70. Accidental pricking with the needle of a used device is thus prevented. Preferably, protective sheath 76 includes a small opening 77 through which the needle travels. As illustrated in FIG. 5A, the sheath spring 70 is extended to automatically extend the protective sheath 76 at the completion of the overall injection.

Sheath spring 70 should have sufficient force to effect the automatic deployment of the protective sheath at the completion of the injection. However, the force provided by spring 70 must be less than the force provided by the drive spring 56. For example, a spring providing a force of 2 to 4 pounds of force may be suitable for the sheath spring. The force of the drive spring 56 must be sufficient to overcome the force of the sheath spring 70 to compress the spring 70 during the injection portion of the stroke and also to drive the syringe and needle for the actual injection.

In accordance with another aspect of the present invention, the protective sheath 76 ratchets forward automatically as it extends upon completion of the dosage portion of the stroke. In FIGS. 5B and 5C, the ratchet structures 90a, 90b engage opposing posts 92 on the protective sheath. The ratchet structure 90 operates similarly to ratchet structure 80. As the protective sheath extends, the posts 92 separate the cantilevered rows 84 of the ratchet structures such that the protective sheath moves downwardly, and is then prevented from moving back upwardly or retracting. As illustrated in FIG. 5B, upon full extension of the protective sheath, the posts 92 will be in their lowermost positions with respect to the corresponding ratchet structure 90 and the sheath is locked. The injector device, once used, can be safely disposed of because the needle is covered and the protective sheath is locked such that it may not easily be pushed back or retracted to expose the needle 50.

In summarizing the operation of the illustrated embodiment of the invention, the injection device 10 may be positioned at an injection site 22. The injection device is then armed, or made ready for firing or actuation by disengaging the safety device, such as post 33, from its engagement with the bifurcated member 58. This arms the injection device 10. Next, the injection device is pushed down so that the exposed portion of the body 18 (FIG. 1A) is pushed up into the housing (FIG. 2A). The bifurcated member 58 is driven against a release device 28, which releases the compressed drive spring 56 to fire the device and drive the syringe and needle for injection (FIG. 3A). The drive member rotates and the syringe ratchets downwardly to its lowermost injection position. Simultaneously, the sheath spring 70 is compressed to thereby arm the protective sheath for operation. At the end of the injection portion of the stroke, the drive member is rotated the desired amount for it to pass through the drive washer and begin driving the plunger. This begins the dosage portion of the stroke as the API is ejected from the syringe. At the end of the dosage portion of the stroke, the drive member engages slide 100 and thereby releases the protective sheath for deployment. As the injection device is withdrawn from the injection site, the sheath automatically deploys and automatically ratchets downwardly to cover the needle 50. The injection device may then be safely discarded.

In accordance with another aspect of the present invention, the protective sheath 76 may be given a unique or bright color, such as forming it from a brightly colored plastic or utilizing a brightly colored decal, to indicate to the user that the device has been used. Alternatively, graphics or symbols might be utilized on the sheath to indicate that it has been used. Therefore, a deployed sheath provides visual information the an injection has been completed and the full dosage amount dispensed.

FIGS. 6A-6C illustrate an alternative embodiment of the invention in which an ergonomically designed housing is utilized with the components of the injection device 10 to provide additional advantages and features. Specifically, referring to FIG. 6A, housing 120 is formed in the shape of what is commonly referred to as a joystick design, rather than as a simple pen design as with housing 20 illustrated in FIG. 1. Housing 120 is contoured and includes a grip 122, which may be formed of a tacky rubber, or rubberized, material for improved security in the hand of a user and more secure positioning of the injection device at an injection site. Grip 122 includes finger indents 124 for the fingers of a user's hand. As illustrated with housing 20 in FIG. 1, the internal components of the injection device fit into the housing 120 generally in a somewhat co-axial fashion, as illustrated in FIG. 1A. As illustrated in FIG. 6A, a portion of sheath 76 extends from the bottom of the housing 120 and the body 14, 18 slides with respect to housing 120 as discussed above. Housing 120 includes an enlarged or widened base section for providing more stable placement of the injection device 110 at an injection site. Housing 120 is designed such that the injection device operates generally similarly with respect to the sequence discussed above. Generally, the housing 120 incorporates alternative safety mechanisms and indicators in accordance with other aspects of the present invention. Specifically, the finger indents 124 position one of the user's fingers, such as the index finger, proximate a safety trigger 130. The safety trigger 130 may be pushed in or indented by the user's finger when the housing 120 is squeezed by the user. Trigger 130 may be suitably and optimally coupled with the safety device, such as device 32, and more specifically mechanically coupled with the post 33 for removing the post 33 from engagement with the bifurcated member upon indentation or actuation of the trigger 130. In that way, engaging the trigger disengages the safety device, and thus arms the injection device 110 for operation. By subsequently positioning the injection device at the injection site and pushing downwardly, such that body 18 pushes up into the housing 120, the injection device may be fired.

As illustrated in FIG. 6B, upon completion of the injection, a safety sheath 76 is deployed from the end of housing 120 to cover the needle. The injection device 110 thus operates very similarly to injection device 10 described above, but utilizes a more ergonomically defined outer housing for a better grip, an alternative arming of the device, and a stabilized base 126 for engagement with the injection site.

In accordance with another aspect of the present invention, an additional visual indicator 140 may be provided on the housing, such as at the top of the housing, such as illustrated in FIG. 6C. The visual indicator 140 may be utilized to indicate that the injection device 110 has been used and, thus, should be discarded. This would be similar to the purpose of the bright color on the sheath 76, as discussed above. To that end, in one embodiment of the indicator 140, a rotating disc 142 might be utilized to rotate within an open window 144 formed in the housing 120. The rotating disc may be operably and mechanically coupled with one or more internal components of the injection device to rotate to the appropriate section, based upon the sequence of the injection cycle. For example, the disc may be divided into multiple sections (for example, three sections) indicated as sections 146a, 146b, and 146c. When section 146a is exposed, it may have suitable colors or graphic thereon to indicate that the injection device 110 has not been used and is in a safe mode or ON SAFE. Upon engaging trigger 130, the internal safety device is disengaged and disc 142 rotates to the section 146b, which has suitable colors, or graphics to indicate that injection device 110 is OFF SAFE and is ready to be fired. Upon pressing the device down at the injection site and firing it, the disc 142 is then rotated to expose section 146c in the window 144, as illustrated in 146c, thus indicating that the device has been used and should be discarded. The exposure of 146c would coincide generally with the extension of the protective sheath 76.

In an alternative embodiment of the invention, only two sections might be utilized on disc 142; one to indicate that the device is unused; and one to indicate that the device has been used and should be discarded. In that way, the device 110 provides additional visual indications of the status of the device in its operational sequence between unused and SAFE to an indication that the device has been used and should be discarded. Housing 120 also provides a large surface 121 at the top of the housing and proximate to window 144 for presenting text or other graphics explaining how the injection device 110 operates.

While the present invention has been illustrated by a description of various embodiments and while these embodiments have been described in considerable detail, it is not the intention of the applicant to restrict or in any way limit the scope of the appended claims to such detail. Additional advantages and modifications will readily appear to those skilled in the art. The invention in its broader aspects is therefore not limited to the specific details, representative apparatus and method, and illustrative examples shown and described. Accordingly, departures may be made from such details without departing from the spirit or scope of applicant's general inventive concept.

1. An injector device comprising:
a body containing a syringe with a needle and plunger;
a drive spring coupled with the syringe and operable, when released, to drive the syringe forward to inject the needle and subsequently to dispense a dosage from the syringe, the drive spring being initially locked in an unreleased position;
a housing containing the body and drive spring;
a release apparatus coupled with the housing;
the body slidable with respect to the housing and configured for sliding upward in the housing when the injector device is pressed down at an injection site to engage the release apparatus and release the drive spring for delivering a dosage.

2. The injector device of claim 1 wherein the body includes a drive assembly including the drive spring and a drive member, the drive member being initially locked for locking the drive spring in the unreleased position.

3. The injector device of claim 2 wherein the drive member is bifurcated and separated for being locked.

4. The injector device of claim 3 wherein the release apparatus is configured to squeeze the bifurcated member together when the body slides with respect to the housing to thereby release the drive spring.

5. The injector device of claim 4 wherein the bifurcated member includes a cam surface that engages a corresponding cam surface of the release apparatus when the body slides in the housing.

6. The injector device of claim 1 further comprising a release spring coupled with the release apparatus for biasing the body downwardly away from the release apparatus.

7. The injector device of claim 3 further comprising a safety for engaging the bifurcated member and maintaining it in a separated condition to prevent release of the drive spring.

8. The injector device of claim 2 wherein the drive member is coupled with the syringe, the drive spring operable for driving the drive member to dispense a dosage from the syringe.

9. The injector device of claim 2 wherein the drive member has a movement stroke, the drive member, in a stroke first portion, driving the syringe to inject the needle and, in a stroke second portion, driving the plunger in the syringe to dispense a dosage.

10. The injector device of claim 9 further comprising a drive washer engaging the syringe, the drive member driving the drive washer and syringe in the stroke first portion and passing through the washer to drive the plunger in the stroke second portion.

11. The injector device of claim 1 wherein the housing is contoured to fit a hand.

12. The injector device of claim 1 wherein the housing includes finger indents.

13. The injector device of claim 7 further comprising a trigger, the trigger being coupled with the safety for disengaging the safety from the bifurcated member.

14. An injector device comprising:
a syringe with a needle and plunger;
a drive member coupled with the syringe and having a stroke to drive the syringe forward to inject the needle and subsequently to drive the plunger forward to dispense a dosage from the syringe;
the drive member rotating from a first position to a second position in the stroke to drive the syringe and needle forward and, upon reaching the second position, driving the plunger forward to dispense a dosage.

15. The injector device of claim 14 further comprising:
a body containing the drive member and syringe;
a slot formed in the body to wrap at least partially around the body between the first and second positions;
the drive member including a key structure for following the slot during rotation between the first and second positions.

16. The injector device of claim 15 wherein the slot, at the second position, extends generally straight along the body, the drive member following the straight slot to drive the plunger forward.

17. The injector device of claim 14 further comprising:
a drive washer positioned between the drive member and syringe;
the drive member, between the first and second positions, driving the washer to drive the syringe, and at the second position, passing through the drive washer to drive the plunger.

18. The injector device of claim 17 wherein the drive washer and drive member have key structures thereon;
the drive member driving the washer to drive the syringe and, at the second position, the key structures aligning so the drive member passes through the drive washer to drive the plunger.

19. The injector device of claim 17 wherein the drive washer includes an aperture therein, the drive member being shaped to not pass through the aperture in the first position but in the second position, to pass through the aperture to drive the plunger.

20. The injector device of claim 19 wherein the drive washer aperture has a cross sectional shape, a section of the drive member having a cross sectional shape corresponding to the cross sectional shape of the aperture to pass through the aperture in the second position.

21. The injector device of claim 14 further comprising a body containing the syringe and drive member, the body including a ratchet structure, the syringe ratcheting in the ratchet structure as it is driven.

22. The injector device of claim 17 further comprising a body containing the syringe and drive member, the body including a ratchet structure, the drive washer ratcheting in the ratchet structure as it is driven.

23. An injector device comprising:
a syringe with a needle and plunger;
a drive member coupled with the syringe and having a stroke to drive the syringe forward to inject the needle and subsequently to drive the plunger forward to dispense a dosage from the syringe;
a drive washer positioned between the drive member and syringe;
the drive member, in an injection portion of the stroke, driving the washer to drive the syringe, and in a dosage portion of the stroke, passing through the drive washer to drive the plunger.

24. The injector of claim 23 wherein the drive washer and drive member have key structures thereon;
the drive member driving the washer to drive the syringe and, in the dosage portion of the stroke, the key structures aligning so the drive member passes through the drive washer to drive the plunger.

25. The injector device of claim 23 wherein the drive washer includes an aperture therein, the drive member being shaped to not pass through the aperture in the first position but in the second position, to pass through the aperture to drive the plunger.

26. The injector device of claim 25 wherein the drive washer aperture has a cross sectional shape, a section of the drive member having a cross sectional shape corresponding to the cross sectional shape of the aperture to pass through the aperture in the second position.

27. The injector device of claim 23 further comprising a body containing the syringe and drive member, the body including a ratchet structure, the drive washer ratcheting in the ratchet structure as it is driven.

28. An injector device comprising:
a syringe with a needle and plunger;
a drive member coupled with the syringe and having a stroke;
the drive member, in an injection portion of the stroke, driving the syringe forward to inject the needle and, in a dosage portion of the stroke, driving the plunger forward to dispense a dosage from the syringe;
a protective sheath;
the protective sheath being biased forwardly during the injection portion and automatically released during the dosage portion to cover the needle when the injection is complete.

29. The injection device of claim 28 wherein the protective sheath is automatically released generally upon completion of the dosage portion.

30. The injection device of claim 28 further comprising a sheath spring, the sheath spring being compressed to bias the sheath during the injection portion of the stroke.

31. The injection device of claim 28 further comprising a latch structure configured to engage the forwardly biased protective sheath to prevent it from being released.

32. The injection device of claim 31 wherein syringe, drive member and sheath are contained in a body, the latch structure being formed in the body.

33. The injection device of claim 31 wherein the movement of the drive member in the dosage portion of the stroke causes the latch structure to release the protective sheath.

34. The injection device of claim 31 wherein the protective sheath includes a tab structure that is engaged by the latch structure.

35. The injection device of claim 33 further comprising a slide coupled with the drive member, the slide moving along the injection device with the drive member to release the protective sheath.

36. The injection device of claim 33 further comprising a slide coupled with the drive member, the slide moving along the injection device with the drive member to engage the latch structure and release the protective sheath.

37. The injection device of claim 36 wherein the slide engages the latch structure generally upon completion of the dosage portion.

38. An injector device comprising:
a syringe with a needle and plunger;
a drive member coupled with the syringe and having a stroke;
the drive member, in an injection portion of the stroke, driving the syringe forward to inject the needle and, in a dosage portion of the stroke, driving the plunger forward to dispense a dosage from the syringe;
a protective sheath;
the protective sheath automatically ratcheting forwardly generally upon completion of the dosage portion of the stroke to cover the needle when the injection is complete.

39. The injection device of claim 38 wherein the protective sheath is prevented from being released during the injection portion of the stroke.

40. The injection device of claim 38 is automatically released generally upon completion of the dosage portion.

41. The injection device of claim 38 further comprising a latch structure configured to engage the protective sheath to prevent it from being released during the injection portion of the stroke.

42. The injection device of claim 41 wherein the movement of the drive member in the dosage portion of the stroke causes the latch structure to release the protective sheath.

43. The injection device of claim 41 wherein the protective sheath includes a tab structure that is engaged by the latch structure.

44. The injection device of claim 38 further comprising a ratchet structure, the protective sheath engaging the ratchet structure to automatically ratchet forwardly.

45. The injection device of claim 43 further comprising a ratchet structure, the protective sheath tab structure engaging the ratchet structure to automatically ratchet forwardly.

46. An injector device comprising:
a body containing a syringe with a needle and plunger;
a drive apparatus coupled with the syringe and operable, when released, to drive the syringe forward to inject the needle and subsequently to dispense a dosage from the syringe, the drive spring being initially locked in an unreleased position;
a housing containing the body and drive apparatus;
a release apparatus coupled with the housing;
the body slidable with respect to the housing and configured for sliding upward in the housing when the injector device is pressed down at an injection site to engage the release apparatus and release the drive apparatus for delivering a dosage.

47. An injector device comprising:
a syringe with a body, a needle and plunger movable in the body;
a drive system coupled with the syringe and having a stroke to drive the syringe body forward to inject the needle and subsequently to drive the plunger forward to dispense a dosage from the syringe;
the drive system, in an injection portion of the stroke, engaging the syringe body to drive the syringe, and in a dosage portion of the stroke, disengaging from the syringe body to drive the plunger.

48. An injector device comprising:
a syringe with a needle and plunger;
a drive system coupled with the syringe and having a stroke;
the drive system, in an injection portion of the stroke, driving the syringe forward to inject the needle and, in a dosage portion of the stroke, driving the plunger forward to dispense a dosage from the syringe;
a protective sheath operable for automatically ratcheting forwardly generally upon completion of the dosage portion of the stroke to cover the needle when the injection is complete.

49. An injector device comprising:
a syringe with a needle and plunger;
a drive system coupled with the syringe and having a stroke;
the drive system, in an injection portion of the stroke, driving the syringe forward to inject the needle and, in a dosage portion of the stroke, driving the plunger forward to dispense a dosage from the syringe;
a protective sheath operable for being biased forwardly during the injection portion and automatically released during the dosage portion to cover the needle when the injection is complete.

50. An injector device comprising:
a body containing a syringe with a needle and plunger;
a drive apparatus coupled with the syringe and operable, when released, to drive the syringe forward to inject the needle and subsequently to dispense a dosage from the syringe, the drive spring being initially locked in an unreleased position;
a housing containing the body and drive apparatus;
a visual indicator coupled with the housing, the visual indicator operable for indicating the status of the injector device.

51. The injector device of claim 50 wherein the visual indicator indicates that the injector device is at least one of ON SAFE, OFF SAFE, or USED.

52. The injector device of claim 50 wherein the visual indicator includes a window that indicates the status of the injector device.

53. The injector device of claim 52 wherein the visual indicator includes a rotating disc, which rotates in the window for indicating the status of the injector device.
